(19) United States
(12) Patent Application Publication
(10) Pub. No.: US 2024/0425884 A1
(43) Pub. Date: Dec. 26, 2024

(54) EFFICIENT HIGH-THROUGHPUT ELECTROPORATION FOR EV AND EXOSOME CARGO LOADING

(71) Applicant: University of Florida Research Foundation, Incorporated, Gainesville, FL (US)

(72) Inventor: Mei HE, Gainesville, FL (US)

(21) Appl. No.: 18/683,545

(22) PCT Filed: Sep. 7, 2022
(86) PCT No.: PCT/US2022/076005

§ 371 Date: Feb. 14, 2024

Related U.S. Application Data
(60) Provisional application No. 63/241,185, filed on Sep. 7, 2021.

(51) Int. Cl. C12N 15/88; A61K 9/50; A61K 48/00; C12M 1/42; C12M 3/06

(52) U.S. Cl. CPC C12N 15/88; A61K 9/5068; A61K 9/5089; A61K 48/0033; C12M 23/16; C12M 35/02

(57) ABSTRACT

Various embodiments provide methods, systems, apparatuses, computer program products, and/or the like for electroporation and cargo loading (e.g., transfection) of EVs and exosomes on a sub-cellular scale. In particular, various embodiments involve the use of droplets that contain EVs/exosomes as well as biomolecular cargo to be loaded into the EVs/exosomes. In various embodiments, the droplets are formed to include the EVs/exosomes and the biomolecular cargo. The droplets are then flowed through a microfluidic channel, which is adjacent and/or interfacing with electrodes configured to generate a uniformly distributed electric potential across the microfluidic channel. The electrodes are specifically configured to cause electric current to affect multiple droplets positioned within the microfluidic channel, such that multiple droplets and their contents may be electroporated at substantially the same time. The droplets may accordingly flow through the microfluidic channel for electroporation.

### CROSS-REFERENCE TO RELATED APPLICATIONS

The present application claims priority to U.S. Provisional Application Ser. No. 63/241,185, filed Sep. 7, 2021, the contents of which are incorporated herein by reference in their entirety.

### ACKNOWLEDGEMENT OF GOVERNMENT SUPPORT

This invention was made with government support under R35 GM133794 awarded by the National Institutes of Health. The government has certain rights in the invention.

### TECHNOLOGICAL FIELD

Various embodiments relate to highly efficient cargo loading into extracellular vesicles and exosomes using electroporation. Some example embodiments may further relate to and/or be applied to drug delivery and therapeutic methods.

### BACKGROUND

Extracellular vesicles (EVs) and exosomes have been emerging in developing drug delivery and therapeutics, due at least in part to their natural biocompatibility, fast cellular uptake, and receptor mediated specific tissue targeting. EVs and exosomes have a critical role of penetrating biological barriers, making them ideally suited as an advanced drug delivery platform. For example, compared to the cell therapy, EVs have shown longer storage stability, which allows safe transportation and delayed therapeutic use. Further, the administration of EVs could avoid the aggregating or clumping in the injured microcirculation that generally occur with cell therapy. EVs also are the cell free system, which avoids the risk of mutagenicity and oncogenicity often from cell therapy.

However, existing methods of loading cargos (e.g., biomolecules, drugs) into nano-sized EVs and exosomes, including cell extrusion, sonication, freeze-thaw cycles, and incubation with membrane permeabilizers, are challenging, inefficient, and time-consuming. For example, while the methods for the production of GMP-grade EVs in recent years have been substantially improved in line with the cellular cultivation system, the successful applications of EVs in therapeutics are entirely dependent on the capacity of cargo loading. The effective approaches to load therapeutic nucleic acid cargos into EVs include: 1) modification of parent cells, e.g., through genetic engineering or medication with cytotoxic drugs; 2) active cargo-loading methods for EVs such as sonication, freeze-thaw cycles, electroporation, sonication, direct transfection, and incubation with membrane permeabilizers. Unfortunately, those passive loading methods suffer from long incubation times and low loading capacity. The most cargo loading efficiency is still quite low (˜30%) among all the approaches. Additionally, EVs released from cells are usually in a limited quantity with dynamic molecular contents.

Thus, there is a need for fast, high-throughput, and highly-efficient cargo loading.

### BRIEF SUMMARY

Electroporation refers to techniques using an electric field to facilitate entry of cargo molecules, including macromolecules, into cells or membrane vesicles. In electroporation, the electric field reversibly increases permeability of the cells or membrane vesicles to the cargo molecules without killing or destroying the cell or membrane vesicle. In example instances, electroporation can be applied to transfect cells, or introducing nucleic acids into cells via the cell membranes with increased permeability.

Various embodiments provide methods, systems, apparatuses, computer program products, and/or the like for electroporation and cargo loading (e.g., transfection) of EVs and exosomes on a sub-cellular scale. While EVs and exosomes may have similar lipid bilayer membranes as those of cells, the reduced size and scale of EVs and exosomes introduce compatibility issues and cause aggregation and low loading efficiency when using conventional electroporation and transfection techniques commonly used with cells. Further, various embodiments provide improvements to the efficiency and throughput of electroporation and cargo loading (e.g., transfection), such that a large amount of loaded EVs/exosomes may be generated and extracted efficiently. That is, various embodiments enable efficient and high-throughput loading of biomolecular and/or biochemical cargo into EVs/exosomes, and in various instances, loaded (e.g., transfected) EVs/exosomes can then be used in applications including drug delivery and therapeutic methods. The efficiency and high-throughput provided by various embodiments then enables implementation in therapeutic development and scaling of drug manufacturing.

In particular, various embodiments involve the use of droplets that contain EVs/exosomes as well as biomolecular cargo to be loaded into the EVs/exosomes. In various embodiments, the droplets are first formed to include the EVs/exosomes and the biomolecular cargo. The droplets are then flowed through a microfluidic channel, which is adjacent and/or interfacing with electrodes configured to generate a uniformly distributed electric potential across the microfluidic channel. In some embodiments, the electrodes are configured to cause electric current to pass through multiple droplets positioned within the microfluidic channel, such that the multiple droplets and their contents are electroporated efficiently and at substantially the same time. The droplets then may pass through the microfluidic channel for electroporation, and EVs/exosomes that are loaded due to the electroporation are then harvested or extracted.

EVs/exosomes amendable cargo loading or electroporation using the described systems and methods can be from any cell type the produces EVs and/or exosomes. In some embodiments, the cell is a mammalian cell, such as, but not limited to, a human cell. In some embodiments, the cell is an immune cell. The immune cell can be, but is not limited to, a dendritic cell or a B cell. The EVs/exosomes can be obtained from cell culture medium or a body fluid. The body fluid can be a mammalian body fluid. The mammalian body fluid can be, but is not limited to human body fluid. The body fluid can be, but is not limited to, plasma, urine, a tissue fluid, saliva, or milk. In some embodiments, the EVs/exosomes are obtained from a plant or plant cell. In some embodiments, the EVs/exosomes are obtained from a bacterium.

The cargo loaded into the EVs/exosomes using the described systems and methods can be, but is not limited to, a targeting molecule or a therapeutic molecule. The therapeutic molecule can be, but is not limited to, a protein, a nucleic acid, or a small molecule. The protein can be, but is not limited to, an antibody, a cytokine, a growth factor, a hormone, or an enzyme. The nucleic acid can be, but is not limited to, a DNA, an RNA, an mRNA, an antisense oligonucleotide, an interfering RNA (siRNA), a nucleic acid encoding a peptide, or a CRISPR RNA.

The cargo loaded EVs/exosomes made using the described systems and methods can be administered to a subject to treat a disease or condition. Treating a condition includes, but is not limited to, gene therapy, immunotherapy, or regenerative therapy.

### BRIEF DESCRIPTION OF THE SEVERAL VIEWS OF THE DRAWING(S)

Having thus described embodiments of the present disclosure in general terms, reference will now be made to the accompanying drawings, which are not necessarily drawn to scale.

FIG. 1 is an overview of an example system architecture for microfluidic droplet-based electroporation system (μDES) for EV/exosome cargo loading, in accordance with embodiments of the present disclosure.

FIG. 2 is an exemplary schematic diagram of an example exosome transfection platform that may be configured with apparatuses, devices, structures, components, and/or the like for microfluidic droplet-based electroporation system (μDES) for EV/exosome cargo loading, according to one embodiment.

FIG. 3 is a block diagram of an exemplary apparatus that may perform various operations for microfluidic droplet-based electroporation system (μDES) for EV/exosome cargo loading, according to one embodiment.

FIG. 4 is an exemplary flowchart of an example process including various operations for microfluidic droplet-based electroporation for EV/exosome cargo loading, according to an example embodiment.

FIGS. 5A-5B provide exemplary diagrams illustrating the generation of microfluidic droplets comprising EVs/exosomes and biomolecular cargo to be loaded into the EVs/exosomes, according to an example embodiment.

FIG. 6 is an exemplary diagram illustrating microfluidic droplets comprising EVs/exosomes and biomolecular cargo being caused to flow within a microfluidic channel adjacent to and/or interfacing with electrodes, according to an example embodiment.

FIGS. 7A-B provide exemplary diagrams illustrating the flow of electric current caused by the electrodes across a microfluidic channel and droplets positioned within the microfluidic channel, according to an example embodiment.

FIG. 8 provides exemplary diagrams illustrating electric potential and the flow of electric current near and across a microfluidic channel and droplets positioned within the microfluidic channel, according to an example embodiment.

FIG. 9 provides exemplary diagrams illustrating electric current density and electric potential for a microfluidic channel and droplets positioned within the microfluidic channel, according to an example embodiment.

FIG. 10 provides an exemplary diagram illustrating a workflow for generating electro-transfected clustered regularly interspaced short palindromic repeats (CRISPR) EVs using microfluidic droplet electroporation device (μDES), according to an example embodiment.

FIG. 11 provides an exemplary diagram illustrating an engineering design of a microfluidic droplet electroporation device (μDES), according to an example embodiment.

FIG. 12A is an exemplary diagram illustrating a picture of a Polydimethylsiloxane (PDMS) fabricated device from a 3-dimension printed mold, according to an example embodiment.

FIG. 12B is an exemplary diagram illustrating a picture of a continuous-flow microfluidic droplet electroporation system (μDES) for harvesting cargo loaded EVs, according to an example embodiment.

FIG. 12C is an exemplary diagram illustrating a picture of droplets generated via a microfluidic device for producing water in oil droplets in a uniform size, according to an example embodiment.

FIG. 12D is an exemplary diagram illustrating a fluorescence microscopic image showing the HEI-OC1 hair cells derived EVs labeled with DiR green fluorescence dye and encapsulated in the droplets for electroporation, according to an example embodiment.

FIG. 12E is an exemplary diagram illustrating a microscopic image of EV encapsulated droplets with a diameter about 600 μm, according to an example embodiment.

FIG. 12F is an exemplary diagram illustrating a microscopic image of EV encapsulated droplets with a diameter about 300 μm, according to an example embodiment.

FIG. 12G is an exemplary COMSOL simulation illustrating a uniform electric field for electroporation of continuous flow droplets in microfluidic channel, according to an example embodiment.

FIG. 12H is an exemplary COMSOL simulation illustrating a uniform electric field over microfluidic droplet electroporation in contrast to non-uniform bulk volume electroporation, according to an example embodiment.

FIG. 13 is an exemplary diagram illustrating optimized electroporation conditions, according to an example embodiment.

FIG. 14A provides an exemplary diagram illustrating a nanoparticle tracking analysis (NTA) of sizes and concentrations from HEI-OC1 EVs after μDES transfection, compared with conventional Neon system transfected EVs, and native EVs without electro-transfection, according to an example embodiment.

FIG. 14B provides an exemplary diagram illustrating a zeta potential analysis of μDES transfected EVs compared with conventional Neon system transfected EVs and native EVs without electro-transfection, according to an example embodiment.

FIG. 14C provides an exemplary diagram illustrating a recovery rate of μDES transfected EVs compared with conventional Neon system transfected EVs, according to an example embodiment.

FIG. 14D provides an exemplary diagram illustrating a transfection rate of μDES transfected EVs compared with conventional Neon system transfected EVs, according to an example embodiment.

FIG. 14E provides an exemplary diagram illustrating transmission electron microscopy (TEM) images showing the morphology of HEI-OC1 EVs before electroporation and after μDES electroporation, according to an example embodiment.

### DETAILED DESCRIPTION

Various embodiments of the present disclosure now will be described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. The terms “approximately” and “substantially” are used herein to refer to being within appropriate manufacturing and/or engineering tolerances. Like numbers refer to like elements throughout.

### I. General Overview and Exemplary Technical Advantages

Various embodiments generally relate to microfluidic droplet-based electroporation for EV/exosome cargo loading. Droplets are generated to include a plurality of EVs and exosomes and also include biomolecular cargo to be loaded into the EVs and exosomes. In example embodiments, the biomolecular cargo may include, but is not limited to, oligonucleotides, nucleic acids, proteins, amino acids, carbohydrates, biomolecular or biochemical compounds, drugs, therapeutic molecules, active pharmaceutical ingredients, targeting ligands, and/or the like. The droplets are then flowed through a microfluidic channel and are subjected to electroporation while positioned within the microfluidic channel. The electroporation is caused by electrodes adjacent to and interfacing with the microfluidic channel, and multiple droplets positioned within the microfluidic channel can be electroporated. In some embodiments, the flow of droplets through the microfluidic channel is continuous. In a continuous flow system, droplets are generated and flowed into the microfluidic chamber and electroporated in a continuous manner. As the droplets containing electroporated EVs/exosomes exit the microfluidic chamber, additional droplets are flowed into the microfluidic chamber. Electroporated EVs/exosomes may also be harvested (extracted) on a continuous basis. The described EV/exosome transfection platform 130 and associated methods and operations provide for continuous flow in generation of droplets, electroporation of the EVs/exosomes, and harvesting of the electroporated EVs/exosomes. Continuous flow can provide for increased throughput, and processing on a scale of milliliters to liters of droplets. In contrast, existing processes handle only microliters to milliliters of EVs/exosomes and are not continuous. Thus, various embodiments describe a microfluidic droplet-based electroporation method for efficient and high-throughput cargo loading into EVs and exosomes. In particular, microfluidic channels, microfluidic platforms, microfluidic subsystems and systems, and/or the like circumvent existing drawbacks relating to low efficiency by providing efficient micro-scale (e.g., subcellular) mass transport.

In particular, various embodiments are uniquely adapted for cargo loading (e.g., transfection) of EVs and exosomes on a subcellular scale and address technical challenges that arise from adapting cellular scale electroporation for cells to a subcellular scale. In particular, such adaptation of cellular scale electroporation to a subcellular scale for EVs and exosomes may be inefficient. For example, cuvette-based electroporation and/or other cellular scale electroporation techniques are inefficient when applied to EVs and exosomes, thereby requiring significantly increased amounts of EVs/exosomes and/or significantly increased amounts of cargo for useful applications. In contrast, described herein are various embodiments that provide electroporation and transfection techniques that are successfully and efficiently performed with reasonable doses of EVs/exosomes and cargo due in part to the application of microfluidics and droplets.

In general, various embodiments provide other technical advantages. For example, flow of droplets comprising EVs/exosomes using microfluidics (e.g., flowing droplets through microfluidic channels) enables improved 3-dimensional and customized geometric control of EVs/exosomes during an electroporation and cargo loading (e.g., transfection) process. Further, various embodiments circumvent potential contamination issues that may arise during electroporation or microfluidic flow, as the EVs/exosomes and biomolecular cargo are enveloped in droplets. Similarly, the EVs/exosomes maintain integrity (e.g., of surface features) and are relatively protected from harm, such as heat from the electrodes. Even further, various embodiments efficiently generate and distribute electric potentials and electric currents for electroporation of droplets, such that a much lower voltage (e.g., 10 volts) is required compared to existing techniques (e.g., on a scale of kilovolts).

### II. Exemplary System Architecture

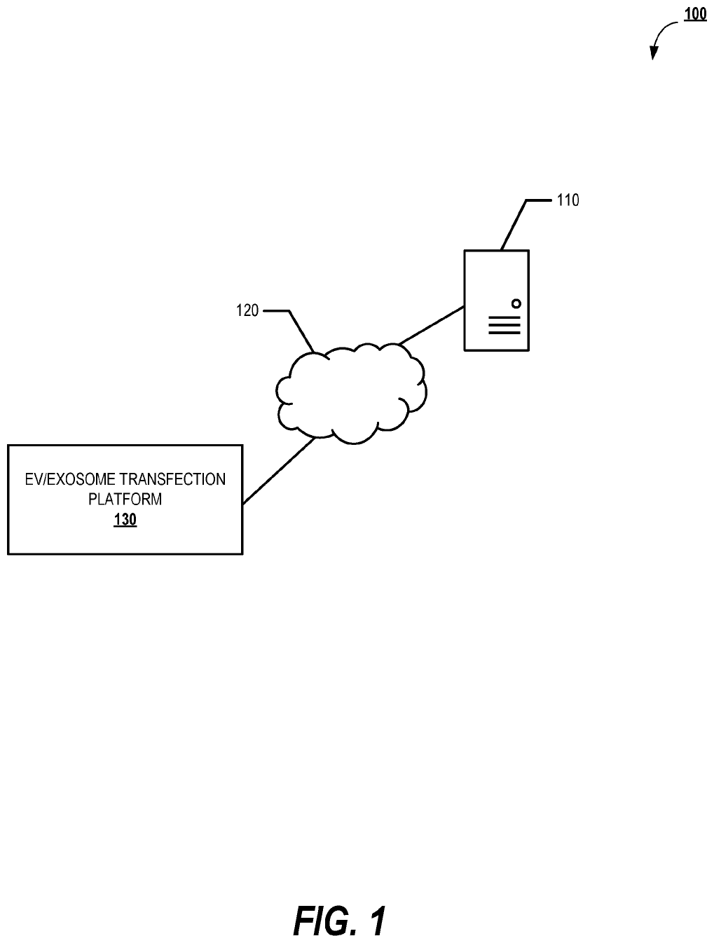

FIG. 1 provides an illustration of an exemplary system architecture 100 according to an example embodiment of the present disclosure. As shown in FIG. 1, the system architecture 100 may include one or more system computing entities 110, one or more networks 120, an EV/exosome transfection platform 130, and/or the like. Each of these components, entities, devices, systems, and similar words used herein interchangeably may be in direct or indirect communication with, for example, one another over the same or different wired or wireless networks 120. In an example embodiment, a system computing entity 110 is in direct wired or wireless communication with the EV/exosome transfection platform 130.

While FIG. 1 illustrates the various system entities as separate, standalone entities, the various embodiments are not limited to this particular architecture. In various embodiments, the system computing entity 110 and the EV/exosome transfection platform 130 may be integrated into a single device or a single system.

In an example embodiment, the system computing entity 110 may be configured to communicate with the EV/exosome transfection platform 130. For example, the system computing entity 110 may communicate with the EV/exosome transfection platform 130 to cause the EV/exosome transfection platform 130 to transfect or otherwise load cargo into EVs and exosomes through microfluidic droplet-based electroporation in accordance with various embodiments described herein. For example, the system computing entity 110 may cause droplets to be generated such that the EVs/exosomes and biomolecular cargo are positioned within the droplets and may cause an electroporation potential to be provided across microfluidic channel through which the droplets flow to cause electroporation of EVs/exosomes in the droplets. In some example embodiments, the system computing entity 110 communicates with the EV/exosome transfection platform 130 to receive data, and the system computing entity 110 may be configured to provide the data (e.g., via display) to a user. For example, the system computing entity 110 may receive sensor data indicating a flow rate of droplets through a microfluidic channel, data indicating an electric field and/or electric currents being provided across a microfluidic channel, and/or the like, and further render at least such sensor data and data for display for a user.

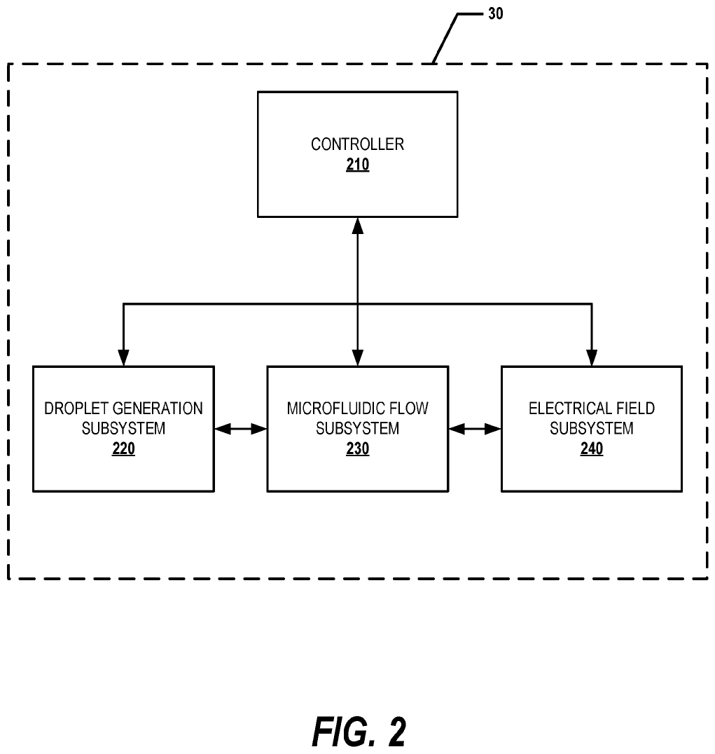

In various embodiments, an EV/exosome transfection platform 130 is configured for microfluidic droplet-based electroporation for EV/exosome cargo loading, such as transfection of EVs/exosomes. As shown in FIG. 2, an example embodiment of the EV/exosome transfection platform 130 comprises a controller 210, a droplet generation subsystem 220, a microfluidic flow subsystem 230, and an electrical field subsystem 240. The controller 210 may be configured to operate and/or control operation of the droplet generation subsystem 220, the microfluidic flow subsystem 230, and the electrical field subsystem 240. For example, the controller 210 specifies a particular EV/exosome, EV/exosome concentration, biomolecular cargo, biomolecular cargo concentration, EV/exosome to biomolecular cargo ratio, and/or buffer to be included in droplets to the droplet generation subsystem 220, a particular flow rate for droplets to the microfluidic flow subsystem 230, and a particular electroporation potential to the electrical field subsystem 240.

In various embodiments, the controller 210 is in communication with the system computing entity 110, and the system computing entity 110 may operate the EV/exosome transfection platform 130 through the controller 210. For example, the system computing entity 110 can transmit a command to the controller 210, and the controller 210 communicates with a particular subsystem of the EV/exosome transfection platform 130 to fulfill the command. Likewise, the controller 210 may aggregate data (e.g., sensor data) from the various subsystems of the EV/exosome transfection platform 130 and provide said data to the system computing entity 110. As previously discussed, the controller and the system computing entity 110 may communicate over a network 120, in various embodiments.

In particular, the droplet generation subsystem 220 may be operated to generate droplets that comprise EVs, exosomes, and/or biomolecular cargo. In doing so, the droplet generation subsystem 220 may be configured for exosome isolation (e.g., exosome extraction, exosome purification, exosome generation, and/or the like), or the droplet generation subsystem 220 is otherwise associated with a subsystem configured to perform exosome isolation. Exosome isolation generally may refer to generation of exosomes, and the exosomes may be generated and extracted from biological material including cell cultures, blood, urine, milk, bacterial fluids, plant fluids, ascites, and/or the like. EVs and exosomes derived from the same cell type may be similar and may include similar surface properties and features. Thus, the droplet generation subsystem 220 (e.g., or an otherwise associated subsystem for exosome isolation) may be configured to generate particular EVs and exosomes from particular cells (e.g., EVs/exosomes from blood, antigen presenting cells, or dendritic cells, liver-type exosomes, cow-muscle exosomes). In some instances, the droplet generation subsystem 220 receives an indication of a particular or selected EV/exosome type (e.g., from the controller 210, from the system computing entity 110) and is configured to isolate and generate EVs/exosomes of the particular or selected EV/exosome type. In many instances, EVs/exosomes naturally include surface features such as antigens and receptors, and thus, EVs/exosomes with a particular antigen may be of interest (e.g., to be loaded with cargo). Thus, the droplet generation subsystem 220 may be configured to store a library of EVs/exosomes and isolate particular EVs/exosomes with a particular antigen, for example. In some embodiments, the droplet generation subsystem 220 is configured to generate droplets based at least in part on an input EV/exosome type and/or biomolecular cargo.

In various embodiments, the droplet generation subsystem 220 may be configured to induce exosome generation in cells of biological material. EVs and exosomes generally are formed based at least in part on endosomes (e.g., multivesicular bodies, intraluminal vesicles) within a cell fusing with the cell membrane of the cell. Thus, the droplet generation subsystem 220 generally may be configured to introduce various hormones, chemicals, compounds, or other stimuli, such as, but not limited to UV light, and/or the like to induce or trigger EV and exosome production or induce or trigger various pathways (e.g., metabolic pathways) that result in generation of EVs and exosomes. The chemical coating reagents introduced to preserve the CRISPR loaded EVs superior stability also result in less membrane fusion and leakage after electro-transfection for utility in clinical settings.

The droplet generation subsystem 220 may further be particularly configured to isolate EVs and exosomes from biological materials, such as blood. Due to the subcellular scale of EVs and exosomes, the droplet generation subsystem 220 may be configured to use centrifugal techniques to isolate EVs and exosomes. Other techniques include ultracentrifugation, microfiltration, chromatography, SELEX, affinity-based purification, antigen-based selection, and/or the like may also be used in isolating EVs and exosomes.

The droplet generation subsystem 220 is further configuration for biomolecular cargo identification, selection, generation, and/or the like. For example, the droplet generation subsystem 220 includes one or more molecular libraries, oligonucleotide libraries, peptide libraries, and/or the like, from which biomolecular cargo may be selected. In drug delivery and therapeutic applications, one or more hormones, proteins, antibodies, nucleic acids, drugs, compounds, therapeutic molecules, active pharmaceutical ingredients, and/or the like, or combinations thereof, may be selected, and the droplet generation subsystem 220 is configured to identify, extract, generate, and/or the like the selected one or more hormones, proteins, antibodies, drugs, compounds, therapeutic molecules, active pharmaceutical ingredients, and/or the like or combinations thereof.

With the EVs/exosomes and biomolecular cargo, the droplet generation subsystem 220 is configured to generate droplets, in various embodiments. The droplets may be understood as microfluidic droplets, emulsion droplets, and/or the like, and thus the droplet generation subsystem 220 is configured to store and use various emulsion fluids, immiscible fluids, and/or the like (e.g., water, buffers, solutions, oils, hexadecane). In various embodiments, the droplet generation subsystem 220 introduces the EVs/exosomes and the biomolecular cargo with a fluid from which the droplets are formed, such that the formed droplets comprise the EVs/exosomes and the biomolecular cargo. The droplet generation subsystem 220 may cause or promote particular interactions between emulsion fluids to cause generation of droplets. For example, the droplet generation subsystem 220 can be configured to use cross-flowing droplet formation, flow-focusing droplet formation, co-flowing droplet formation, and/or the like.

The microfluidic channels of the microfluidic flow subsystem 230 may be configured for flow of droplets generated by the droplet generation subsystem 220. That is, the generated droplets may flow through microfluidic channels of the microfluidic flow subsystem 230. In various embodiments, the microfluidic flow subsystem 230 includes one or more flow rate sensors, temperature sensors, microfluidic channels and microfluidic plates comprising channels, fluid reservoirs and fluid supplies, tubing and piping, and/or the like.

The electrical field subsystem 240 comprises electrodes, and the electrodes of the electrical field subsystem 240 may be specifically positioned on opposite sides of a microfluidic channel (e.g., of the microfluidic flow subsystem 230) through which droplets are configured to flow. In some embodiments, the electrodes interface with the microfluidic channel, are adjacent with the microfluidic channel, are positioned within a threshold distance of the microfluidic channel, and/or the like. In particular, the electrical field subsystem 240 comprises at least one positive electrode and at least one negative electrode, such that electric currents may be provided across the microfluidic channel. In an example embodiment, the electrodes are configured with an electric permittivity of about 20 S/m using a composition of polyaniline hydrogel. In some embodiments, the electrodes are configured with an electric permittivity between about 10 S/m and 30 S/m. In some embodiments, the electrodes are configured with an electric permittivity between about 15 S/m and 25 S/m, between 16 S/m and 24 S/m, or between 18 S/m and 22 S/m. Accordingly, the electrical field subsystem 240 may comprise the electrodes, in addition to various voltmeters, ammeters, multi-meters, resistors, power supplies and converters, voltage supplies and current supplies, voltage and current controllers, and/or the like.

### III. Exemplary Apparatuses

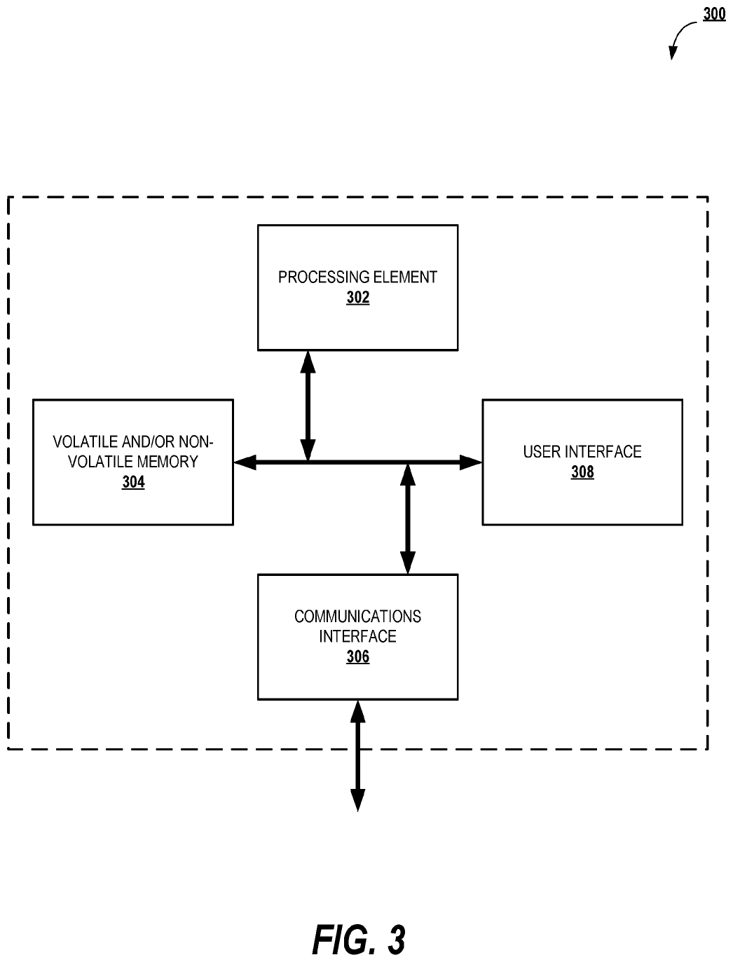

FIG. 3 provides a block diagram of an apparatus 300, according to one embodiment of the present disclosure. In some example embodiments, the apparatus 300 is an embodiment of the system computing entity 110, is embodied by the system computing entity 110, or is otherwise associated with the system computing entity 110. Alternatively, the apparatus 300 may be an embodiment of the controller 210 of the EV/exosome transfection platform 130, may be embodied by the controller 210, or may be otherwise associated with the controller 210. In any regard, the apparatus 300 is configured to perform and/or control various operations for microfluidic droplet-based electroporation for EV/exosome cargo loading. For example, the system computing entity 110 and/or the controller 210 (e.g., when embodied by the apparatus 300) may at least control generation of droplets comprising non-loaded EVs/exosomes and biomolecular cargo, flow of the droplets through a microfluidic channel, generation of a uniformly distributed electric field across the microfluidic channel, and extraction of loaded EVs/exosomes.

In general, the terms computing entity, computer, entity, device, system, and/or similar words used herein interchangeably may refer to, for example, one or more computers, computing entities, desktops, mobile phones, tablets, phablets, notebooks, laptops, distributed systems, input terminals, servers or server networks, blades, gateways, switches, processing elements, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

In one embodiment, the apparatus 300 may also include one or more communications interfaces 306 for communicating with various other computing entities, such as by communicating data, content, information, and/or similar terms used herein interchangeably that can be transmitted, received, operated on, processed, displayed, stored, and/or the like.

As shown in FIG. 3, in one embodiment, the apparatus 300 may include or be in communication with one or more processing elements 302 (also referred to as processors, processing circuitry, and/or similar terms used herein interchangeably) that communicate with other elements within the apparatus 300 via a bus, for example. As will be understood, the processing element 302 may be embodied in a number of different ways. For example, the processing element 302 may be embodied as one or more complex programmable logic devices (CPLDs), microprocessors, multi-core processors, co-processing entities, application-specific instruction-set processors (ASIPs), microcontrollers, and/or controllers. Further, the processing element 302 may be embodied as one or more other processing elements or circuitry. The term circuitry may refer to an entirely hardware embodiment or a combination of hardware and computer program products. Thus, the processing element 302 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 302 may be configured for a particular use or configured to execute instructions stored in volatile or non-volatile media or otherwise accessible to the processing element 302. As such, whether configured by hardware or computer program products, or by a combination thereof, the processing element 302 may be capable of performing steps or operations according to embodiments of the present disclosure when configured accordingly.

In one embodiment, the apparatus 300 may further include or be in communication with memory 304. In an example embodiment, the memory 304 comprises non-volatile media (also referred to as non-volatile storage, memory, memory storage, memory circuitry and/or similar terms used herein interchangeably). In one embodiment, the non-volatile storage or memory may include one or more non-volatile storage or memory media, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the memory 304 may further include or be in communication with volatile media (also referred to as volatile storage, memory, memory storage, memory circuitry and/or similar terms used herein interchangeably). In one embodiment, the volatile storage or memory may also include one or more volatile storage or memory media, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 302. Thus, the databases, database instances, database management systems, data, applications, programs, program modules, scripts, source code, object code, byte code, compiled code, interpreted code, machine code, executable instructions, and/or the like may be used to control certain aspects of the operation of the apparatus 300 with the assistance of the processing element 302 and operating system.

As indicated, in one embodiment, the apparatus 300 may also include one or more communications interfaces 306 for communicating with various other computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the apparatus 300 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

When embodied by the controller 210, the apparatus 300 may communicate with various subsystems, modules, components, devices, and/or the like of the EV/exosome transfection platform 130 using a communications interface 306, in some embodiments. For example, the apparatus 300 transmits commands via communication interface 306 to the microfluidic flow subsystem 230 to control the flow rate of droplets through a microfluidic channel. As another example, the apparatus 300 receives sensor data from the electrical field subsystem via communications interface 306.

The apparatus 300 may also comprise a user interface 308 (that can include a display coupled to a processing element). For example, the user interface 308 may include or be in communication with one or more input elements, such as a keyboard input, a mouse input, a touch screen/display input, motion input, movement input, audio input, pointing device input, joystick input, keypad input, and/or the like. The apparatus 300 may also include or be in communication with one or more output elements (not shown), such as audio output, video output, screen/display output, motion output, movement output, and/or the like. These input and output elements may include software components, such as a user application, browser, graphical user interface, and/or the like to facilitate interactions with and/or cause display of information/data from the apparatus 300, as described herein. The user input interface can comprise any of a number of devices or interfaces allowing the apparatus 300 to receive data, such as a keypad (hard or soft), a touch display, voice/speech or motion interfaces, or other input device. In embodiments including a keypad, the keypad can include (or cause display of) the conventional numeric (0-9) and related keys (#, *), and other keys used for operating the apparatus 300 and may include a full set of alphabetic keys or set of keys that may be activated to provide a full set of alphanumeric keys. For example, a user of the apparatus 300 may configure a particular electric potential, electric current, electric field, microfluidic flow rate, and/or the like for electroporation of droplets comprising EVs/exosomes using the user interface 308. Similarly, the user interface 308 may be provided to display various data for the EV/exosome transfection platform 130 and relevant to the microfluidic droplet-based electroporation of EVs/exosomes. For example, flow rates, droplet count estimates, fluid volumes, electric potentials, electric currents, and/or the like may be displayed via user interface 308 for a user.

As will be appreciated, one or more of the components of the apparatus 300 may be located remotely from other components of the apparatus 300, such as in a distributed system. Furthermore, one or more of these components may be combined with additional components to perform various functions described herein, and these additional components may also be included in the apparatus 300. Thus, the apparatus 300 can be adapted to accommodate a variety of needs and circumstances. As will be recognized, these architectures and descriptions are provided for exemplary purposes only and are not limiting to the various embodiments.

### IV. Exemplary Operations

Example embodiments of the present disclosure provide for operations for microfluidic droplet-based electroporation for EV/exosome cargo loading (e.g., EV/exosome transfection). Example embodiments described herein provide various technical advantages that include improved efficiency, improved throughput of transfection, improved integrity of EVs/exosomes during electroporation, and reduced contamination. Due to the use of microfluidic channels and configuration of electrodes, multiple droplets that are positioned within one or more microfluidic channels can be simultaneously or almost simultaneously subjected to electroporation (e.g., subjected to electric currents configured to cause electroporation). With continuous flow of droplets through the microfluidic channel, throughput and efficiency of EV/exosome cargo loading (e.g., transfection) is significantly improved. Use of EVs/exosomes and biomolecular cargo within droplets can be used to control and/or limit electric currents affecting the EVs/exosomes such that integrity (e.g., of membranes) of the EVs/exosomes can be maintained. The concentration of EVs/exosomes and biomolecular cargo within the droplets, selection of aqueous solution and/or buffer in the droplets, and/or selection of immiscible fluid (e.g., oil) can be altered to optimize electroporation of the EVs/exosomes. Additionally, the use of EVs/exosomes and biomolecular cargo within droplets reduces contamination that may occur with contact with the electrodes.

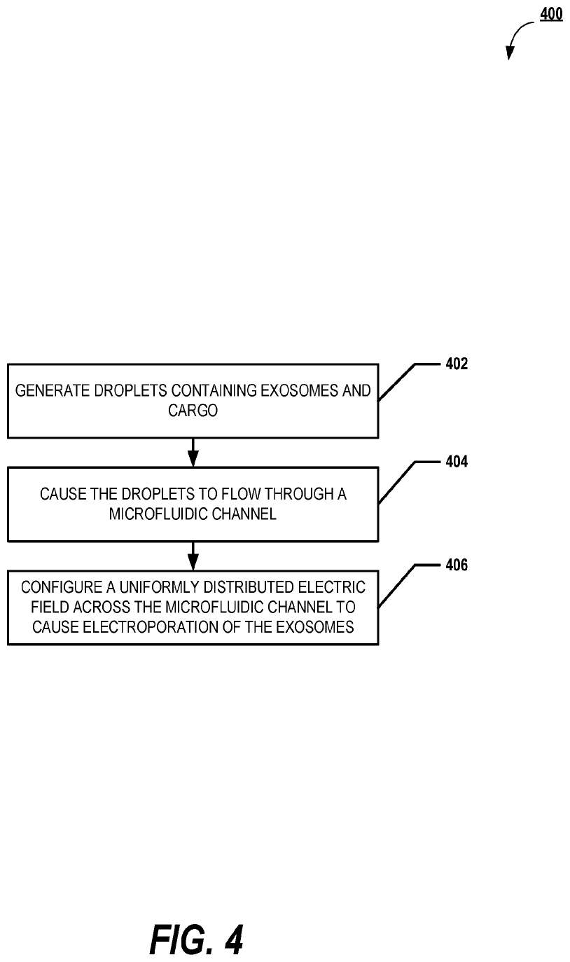

FIG. 4 provides a flowchart of an example process 400 for microfluidic droplet-based electroporation and cargo loading (e.g., transfection) of EVs and exosomes. Process 400 includes example steps/operations that may be performed by apparatus 300, for example, when embodied by system computing entity 110 or when embodied by controller 210 of the EV/exosome transfection platform 130. The apparatus 300 includes means, such as processing element 302, memory 304, communications interface 306, and/or the like, for performing and/or controlling each step/operation of process 400.

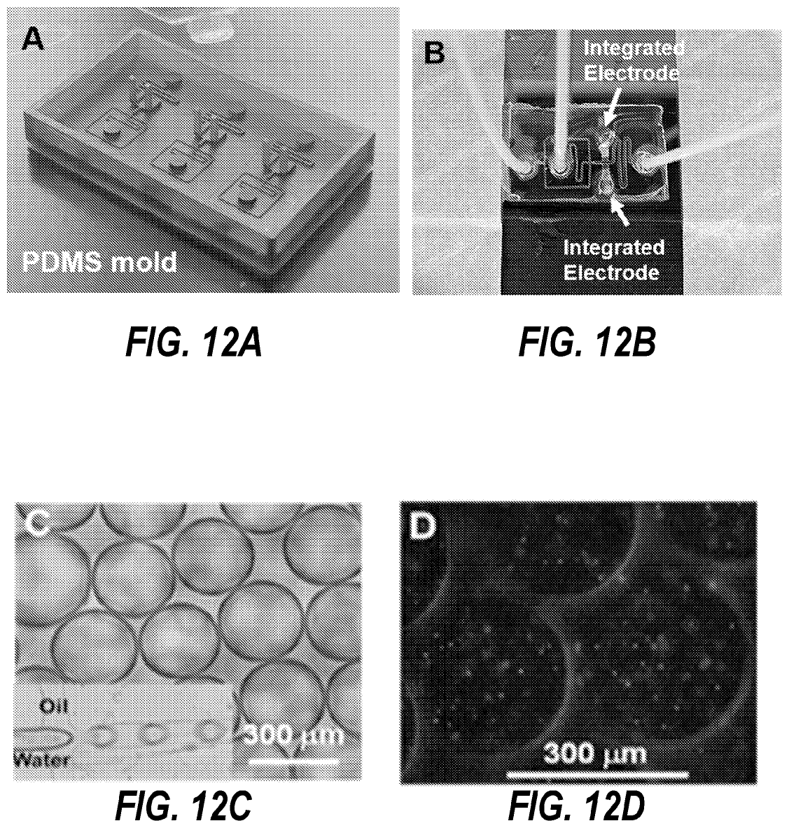

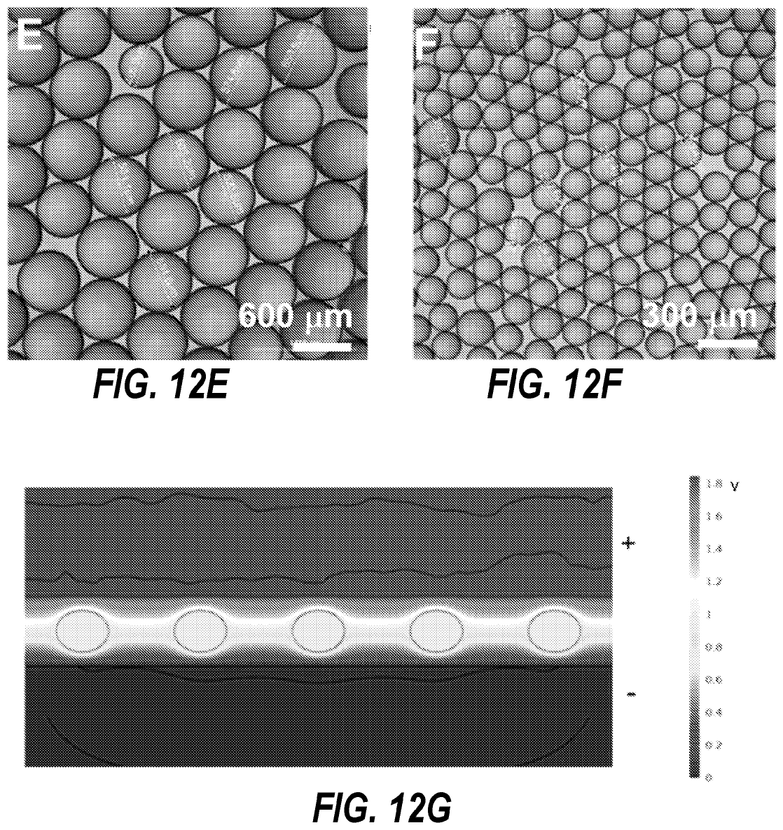

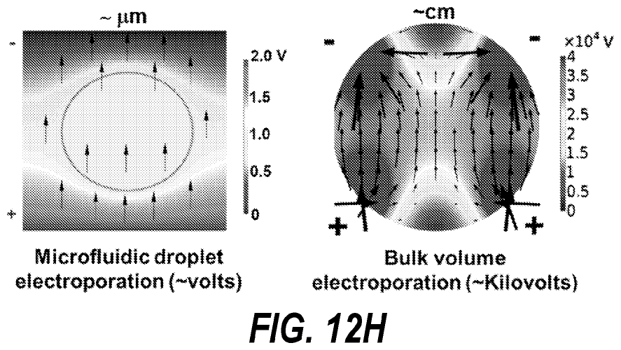

As illustrated, process 400 includes step/operation 402. At step/operation 402, droplets are generated. A droplet refers to a small and discrete volume of fluid that is particularly useful and applicable in microfluidics. The droplets are sized to flow through the microfluidic chamber. In some embodiments, the droplets are sized to have a diameter that corresponds about the internal diameter of the microfluidic chamber. In some embodiments, the droplets are sized to have a diameter that is less than the internal diameter of the microfluidic chamber. In some embodiments, the droplet comprises a fluid that is compatible with the EVs/exosomes. Compatible indicates the EVs/exosomes can survive in the fluid at least for a period of time necessary to perform the droplet generation and electroporation procedure and subsequent extraction. A droplet exists in a fluid environment in an immiscible form. For example, surfactants may be involved in generating droplets. In various embodiments, the droplets are water-in-oil droplets; that is, a droplet comprises a small volume of water and exists in an environment of oil (it is understood that water and oil are immiscible). FIG. 12E is an exemplary diagram illustrating device manipulation to generate droplet size of 650 μm with different EV encapsulation rates for EV electroporation, according to an example embodiment. FIG. 12F is an exemplary diagram illustrating device manipulation to generate droplet size of 300 μm with different EV encapsulation rates for EV electroporation, according to an example embodiment.

A generated droplet comprises a plurality of EVs and/or a plurality of exosomes and further comprises biomolecular cargo to be loaded into the EVs and/or the exosomes. For example, the biomolecular cargo can comprise one or more oligonucleotides, nucleic acids, proteins, amino acids, carbohydrates, biomolecular or biochemical compounds, drugs, therapeutic molecules, active pharmaceutical ingredients, and/or the like or combinations thereof to be delivered by EVs and/or exosomes to a particular tissue in a drug delivery or therapeutic application. In some example instances, one or more particular biomolecular cargoes are selected (e.g., by a user via user interface 308), and the generated droplets specifically comprise the selected biomolecular cargoes (e.g., nucleic acids with specific nucleotide sequences). In various embodiments, the controller 210 performs step/operation 402 such that the droplet generation subsystem 220 generates a plurality of droplets comprising a plurality of EVs/exosomes and a plurality of biomolecular cargo. FIG. 12C is an exemplary diagram illustrating a picture of droplets generated via a microfluidic device for producing water in oil droplets in a uniform size, according to an example embodiment. FIG. 12D is an exemplary diagram illustrating a fluorescence microscopic image showing the HEI-OC1 hair cells derived EVs labeled with DiR green fluorescence dye and encapsulated in the droplets for electroporation, according to an example embodiment. FIG. 12E is an exemplary diagram illustrating a microscopic image of EV encapsulated droplets with a diameter about 600 μm, according to an example embodiment. FIG. 12F is an exemplary diagram illustrating a microscopic image of EV encapsulated droplets with a diameter about 300 μm, according to an example embodiment. FIG. 12G is an exemplary COMSOL simulation illustrating a uniform electric field for electroporation of continuous flow droplets in microfluidic channel, according to an example embodiment. FIG. 12H is an exemplary COMSOL simulation illustrating a uniform electric field over microfluidic droplet electroporation in contrast to non-uniform bulk volume electroporation, according to an example embodiment.

In various embodiments, EVs and/or exosomes may first be generated or extracted. In some embodiments, the EVs and exosomes originate from cells. EVs and exosomes are membranous vesicles typically released by cells, such as, but not limited to, antigen presenting cells. Cell-derived EVs and exosomes typically originate from endosomal compartments and are produced during the vesicular transport from the endoplasmic reticulum (ER) to the Golgi apparatus. EVs and exosomes can be released extracellularly after the multivesicular bodies are fused with the plasma membrane. Thus, generation, extraction, and isolation of EVs and exosomes may involve inducing production of such endosomes within cells and isolating the EVs and exosomes from the cells. Generally, EVs and exosomes can be classified and characterized based at least in part on the cells from which they originate. For example, an exosome originating from a liver cell and an exosome originating from a blood cell may be uniquely different, with different surface characteristics, properties, features, and/or the like. In various embodiments, EVs and exosomes may originate from cell cultures, blood, urine, milk, bacterial fluids, plant fluids, ascites, and/or the like. In particular, EVs and exosomes may include surface features such as antigens, receptors, peptides, and/or the like. Thus, in various embodiments, EVs and exosomes of a particular origin and with particular surface features may be generated. EVs are nanoscale membrane vesicles and typically contain numerous plasma membrane and cytoplasmic components from the cells from which they are derived. In some embodiments, the EVs and exosomes are synthetic.

Similarly, biomolecular cargoes are selected or generated in preparation for the generation of droplets. Biomolecular cargoes, such as drugs, hormones, nucleic acids, and/or the like, can be selected from known therapeutic agents, molecules having known biological properties, various libraries, or generated as needed. For example, a particular biomolecular cargo can be identified, selected, and extracted from oligonucleotide libraries, peptide libraries, and/or the like. A biomolecular cargo can be selected based at least in part on a function or property desired in the EV or exosome. In various embodiments, an indication originating from the system computing entity 110 and/or a user that describes one or more particular or selected biomolecular cargoes is received, and generation of droplets is based at least in part on the one or more particular or selected biomolecular cargoes. In association with the particular or selected biomolecular cargoes, an indication of a target destination or tissue (e.g., such as in drug delivery and therapeutic applications) may be received, and particular EVs and exosomes with surface features configures for the target destination or tissue are generated. In some embodiments, one or more biological cargo molecules are provided to the droplet generation subsystem 220, and the droplet generation subsystem 220 is configured to form droplets containing the one or more biological cargo molecules and EVs/exosomes. In some embodiments, isolated EVs/exosomes are provided to the droplet generation subsystem 220, and the droplet generation subsystem 220 is configured to form droplets containing the isolated EVs/exosomes and one or more selected biological cargo molecules. In some embodiments, isolated EVs/exosomes and one or more biological cargo molecules are provided to the droplet generation subsystem 220, and the droplet generation subsystem 220 is configured form droplets containing the isolated EVs/exosomes and the one or more selected biological cargo molecules.

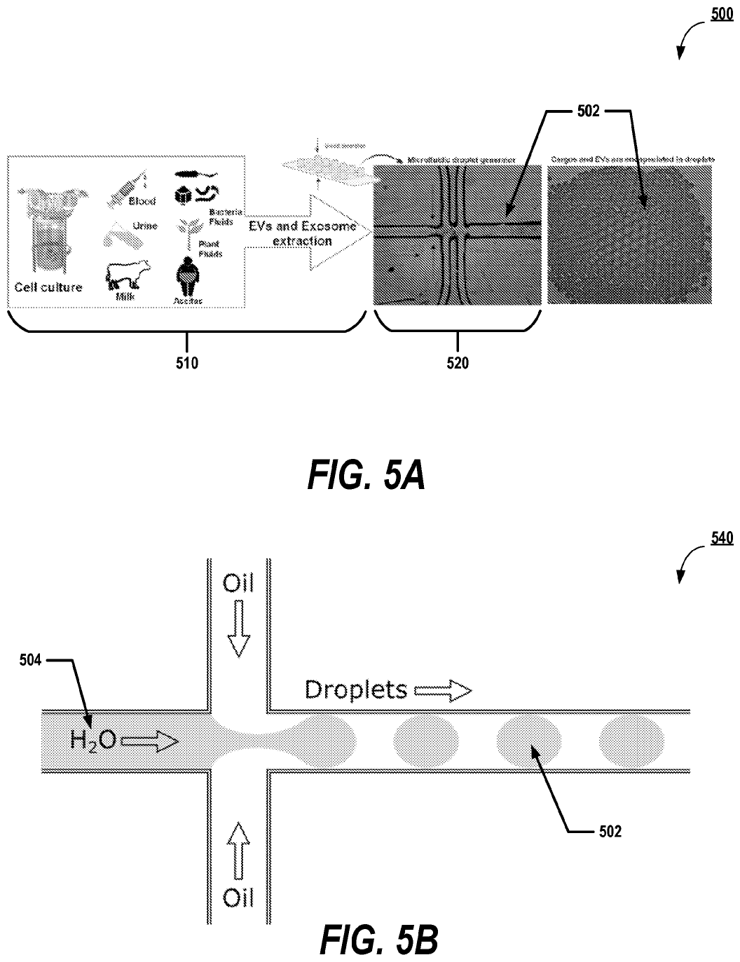

FIG. 5A and FIG. 5B provide diagrams illustrating the generation of droplets comprising EVs/exosomes and biomolecular cargo. Referring first to FIG. 5A, a diagram 500 is provided. As shown in diagram 500, generation of droplets involves EV/exosome extraction 510. As discussed above, EV/exosome extraction 510 may involve at least generation of exosomes from cells and isolation of the exosomes (e.g., via centrifugation, filtration, chromatography, and/or the like). The EVs, exosomes, and the biomolecular cargo are then introduced into a fluid from which droplets are formed.

Diagram 500 then illustrates droplet generation 520. In the illustrated embodiment, droplet generation 520 involves flow-focusing droplet formation specifically, although various other embodiments may involve cross-flowing droplet formation (e.g., using a T-shaped junction, using a Y-shaped junction), co-flowing droplet formation, and/or the like. FIG. 5B provides a diagram 540 specifically illustrating flow-focusing droplet formation. As shown in FIG. 5B, an immiscible fluid 504, such as water, flow through a main channel, and the main channel is intersected by at least two additional channels on opposite sides. While FIG. 5B illustrates one additional channel or inlet on each side of the main channel, other example embodiments may include two additional channels or inlets one each side of the main channel, as illustrated in FIG. 5A. Another fluid (e.g., oil) with which the immiscible fluid 504 is immiscible flows through the at least two additional channels, such that the fluid (e.g., oil) effectively pinches off flow of the immiscible fluid 504 to form droplets 502 of the immiscible fluid 504. As a result, droplets are formed to comprise EVs/exosomes, and biomolecular cargo. In some instances, the fluid is hexadecane, and the droplets are accordingly water-in-hexadecane droplets. In some instances, the immiscible fluid 504 from which the droplets 502 are formed is a phosphate buffered saline (PBS) with a conductivity of about 1 S/m. In some embodiments, the droplets 502 are formed to have a conductivity between about 0.2 S/m and about 2 S/m, a conductivity between about 0.5 S/m and about 1.7 S/m, or a conductivity between 0.7 S/m and 1.5 S/m. In some embodiments, the droplets 502 are formed to have a conductivity of about 0.2 S/m, about 0.3 S/m, about 0.5 S/m, about 0.7 S/m, about 0.8 S/m, or about 0.9 S/m. In some embodiments, the droplets 502 are formed to have a conductivity of about 1.1 S/m, about 1.2 S/m, about 1.3 S/m, about 1.4 S/m, about 1.5 S/m, or about 1.6 S/m. In some embodiments, the conductivity of the droplets 502 is near 0 S/m; for example, the conductivity of the droplets can be 3×10⁻⁷ (3e−7) S/m. In some embodiments, the conductivity of the droplets is between approximately 1e−9 S/m and approximately 1e−4 S/m. In various embodiments, the immiscible fluid 504 comprises the EVs/exosomes and the biomolecular cargo, and thus, droplets 502 are formed to comprise the EVs/exosomes and the biomolecular cargo.

In instances in which the droplet generation subsystem 220 employs or applies flow-focusing droplet formation, a size of the droplets 502 as well as other various parameter and aspects of droplet generation 520 can be configured. Generally, the size of the droplets 502 is related to the flow rate of the fluid (e.g., oil) through the at least two additional channels. Thus, the droplet generation subsystem 220 may be configured to, responsive to commands from controller 210 for example, set a flow rate of the fluid (e.g., oil) through the at least two additional channels to generate droplets 502 of a particular size. In instances in which other droplet formation techniques (e.g., co-flowing droplet formation, cross-flowing droplet formation) are employed, the droplet generation subsystem 220 is configured to use other various mechanisms to control aspects of droplet generation 520 such as droplet size. In various embodiments, the droplets are generated with a size of about 60 micrometers (60 μm). In some embodiments, the droplets are about 40 μm, about 45 μm, about 50 μm, about 55 μm, about 60 μm, about 65 μm, about 70 μm, about 75 μm, or about 80 μm in diameter. In some embodiments, the droplets are about 40 to about 80 μm in diameter, about 45 to about 75 μm in diameter, about 50 to about 70 μm in diameter, or about 55 to about 65 μm in diameter.

Returning now to FIG. 4, process 400 further includes step/operation 404. Process 400 comprises causing the droplets 502 to flow through a microfluidic channel. In some embodiments, the droplets 502 directly flow through the microfluidic channel subsequent to being generated; that is, droplet generation 520 and droplet flow through the microfluidic channel is continuous and connected. In other example embodiments, the droplets 502 are stored or deposited in some reservoir (e.g., filled with oil, hexadecane, and/or the like to preserve immiscibility) before continuing to flow through the microfluidic channel.

The microfluidic channel may be configured with a particular diameter based at least in part on droplet size and may accordingly filter out abnormal droplets (e.g., undesired large droplets). In various embodiments, the microfluidic channel is 1000 micrometers by 1000 micrometers by 100 micrometers (1000 μm×1000 μm×100 μm). In particular, the microfluidic channel has a width of 100 μm through which droplets 502 (e.g., which may be sized based at least in part on the 100 μm width, which may have a diameter of 60 μm) can flow. The microfluidic channel then spans, extends, covers, and/or the like at least a portion of an area of 1000 μm by 1000 μm. In various embodiments, channel geometry is specifically defined and optimized to provide high efficiency and high-throughput of droplet flow through the microfluidic channel. In some embodiments, the width of the microfluidic channel is between about 50 μm and about 200 μm, between about 80 μm and about 150 μm, or between 100 μm and about 120 μm. The length of the microfluidic channel (e.g., the distance that a droplet 502 may travel while flowing through the microfluidic channel) may be between about 35 μm and 5 millimeters (mm). In some embodiments, the length of the microfluidic channel is between about 100 μm and 3 mm, between about 500 μm and 2 mm, or between 1 mm and 1.5 mm.

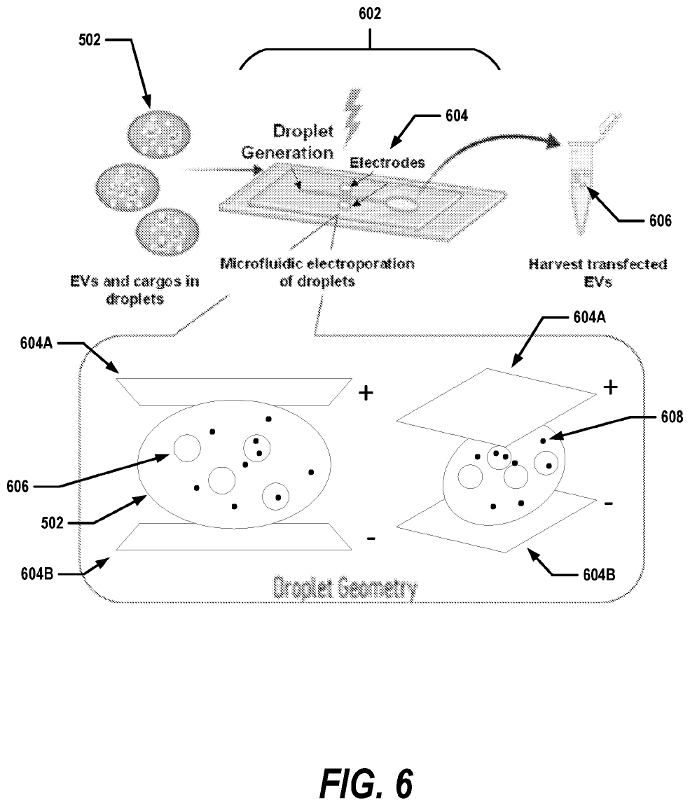

FIG. 6 provides a diagram illustrating introduction of droplets 502 to a microfluidic plate 602 including a microfluidic channel and continuous flow of the droplets 502 through the microfluidic channel. As shown, the droplets 502 comprise EVs/exosomes 606 as well as biomolecular cargo 608. FIG. 6 further illustrates that the microfluidic channel is sandwiched or bordered by at least two electrodes 604. That is, the electrodes 604 are positioned apart by a distance that includes at least the width of the microfluidic channel (e.g., 100 μm, between 50 μm and 150 μm). The electrodes 604 are configured to generate an electric field across the microfluidic channel. Specifically, the electrodes 604 provide an electric potential across the microfluidic channel that generates electric current that affects droplets 502, and the EVs/exosomes 606 positioned within the droplets 502, passing through microfluidic channel, and the electric potential is specifically defined to be an electric potential causing electroporation of the EVs/exosomes 606 stored in the droplets 502. In various embodiments, EVs/exosomes 606 of a particular origin are associated with a respective threshold electroporation potential, and thus, the electrodes 604 provide an electric potential satisfying the threshold electroporation potential of the EVs/exosomes 606. The threshold electroporation potential is the electric potential necessary to enhance uptake of the biomolecular cargo into the EVs/exosomes 606 while retaining activity of the EVs/exosomes post electroporation. The threshold electroporation potential can be determined empirically for a given batch of EVs/exosomes, or determined empirically for EVs/exosomes. For example, EVs/exosomes can be generated and then electroporated with a range of electrical potentials using the described device(s) to determine to optimum electrical potential for the particular EVs/exosomes. In some embodiments, threshold electroporation potential is measured in volts/cm or electric current density. The fluid from which the droplets are formed is associated with a conductivity such that the droplets 502 convey the electric current to the EVs/exosomes 606 positioned within the droplets 502. Thus, the electrodes 604 are generally configured to induce electroporation of the EVs/exosomes 606 such that the biomolecular cargo 608 is loaded into the EVs/exosomes 606.

In various embodiments, the electrodes 604 include a positive electrode 604A and a negative electrode 604B positioned on opposite sides of the microfluidic channel. As a result, droplets 502 flow through the microfluidic channel between the positive electrode 604A and the negative electrode 604B, such that an electric current caused by the positive electrode 604A and the negative electrode 604B affects (e.g., impacts, passes through) the droplets 502 flowing through the microfluidic channel and positioned between the positive electrode 604A and the negative electrode 604B.

Returning to FIG. 4, process 400 further includes step/operation 406. At step/operation 406, the apparatus 300 (e.g., embodied by system computing entity 110, embodied by controller 210) configures a uniformly distributed electric field across the microfluidic channel. The uniformly distributed electric field is configured to cause electroporation of the EVs/exosomes within the droplets 502. As discussed, droplets 502 are formed from a buffer, such as phosphate buffered saline (PBS) with a conductivity of about 1 S/m, thus, electric current from the uniformly distributed electric field affects the EVs/exosomes within the droplets 502. That is, the droplets 502 are generated and configured to convey electric current to EVs/exosomes positioned within the droplets 502.

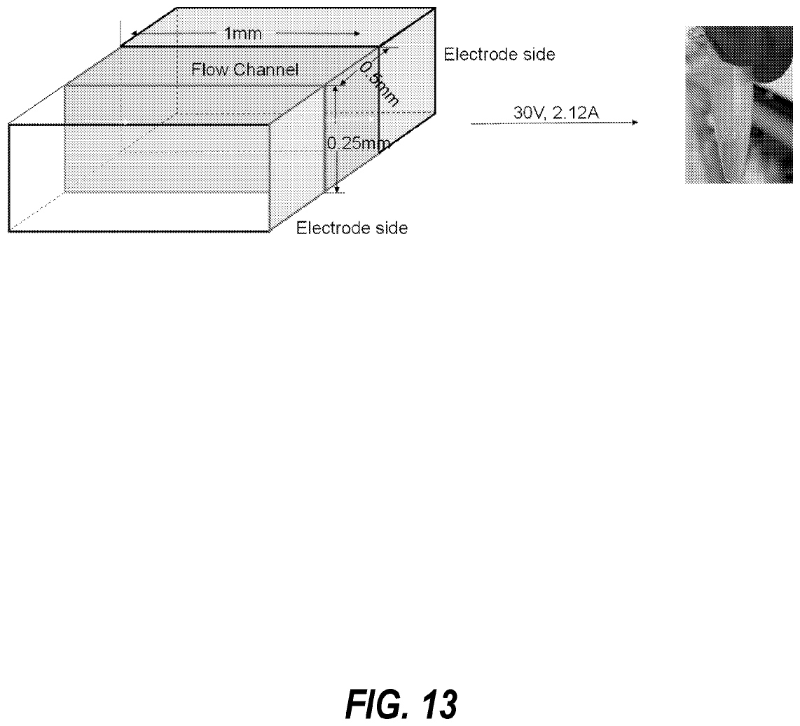

In various embodiments, the uniformly distributed electric field is configured based at least in part on applying a voltage to the electrodes 604, and in one example embodiment, the applied voltage ranges from approximately 10 volts (10 V) to approximately 1850 volts (1850 V). The applied voltage can be configured according to characteristics and properties of the EVs/exosomes 606. In some instances, the EVs/exosomes 606 may require a threshold electroporation potential for hydrophilic pores to appear in their membranes, and the applied voltage is based at least in part on the threshold electroporation potential. In some embodiments, the applied voltage and the uniformly distributed electric field are configured based at least in part on performing various three-dimensional simulations for electric field distribution (e.g., via COMSOL, via finite element analysis). FIG. 13 is an exemplary diagram illustrating optimized electroporation conditions, according to an example embodiment. As shown in FIG. 13, the flow channel and electrode dimension were demonstrated. The continuous flow may allow the collection of droplets into the collection tube for separating EVs in the water phase from the oil phase via centrifugation. The optimized electroporation conditions may include: a flow rate equals about 3.8 uL/sec, an electroporation intersection equals about 1×0.5×0.25 mm³, and a time duration equals about 0.032 sec. After electroporation, EV in the droplets may be easily obtained in the tube for membrane recovery. It takes 6 minutes to fully separate an oil phase from an aqueous phase with a centrifuge at 200 xg, 500 xg, and 2000 xg, respectively.

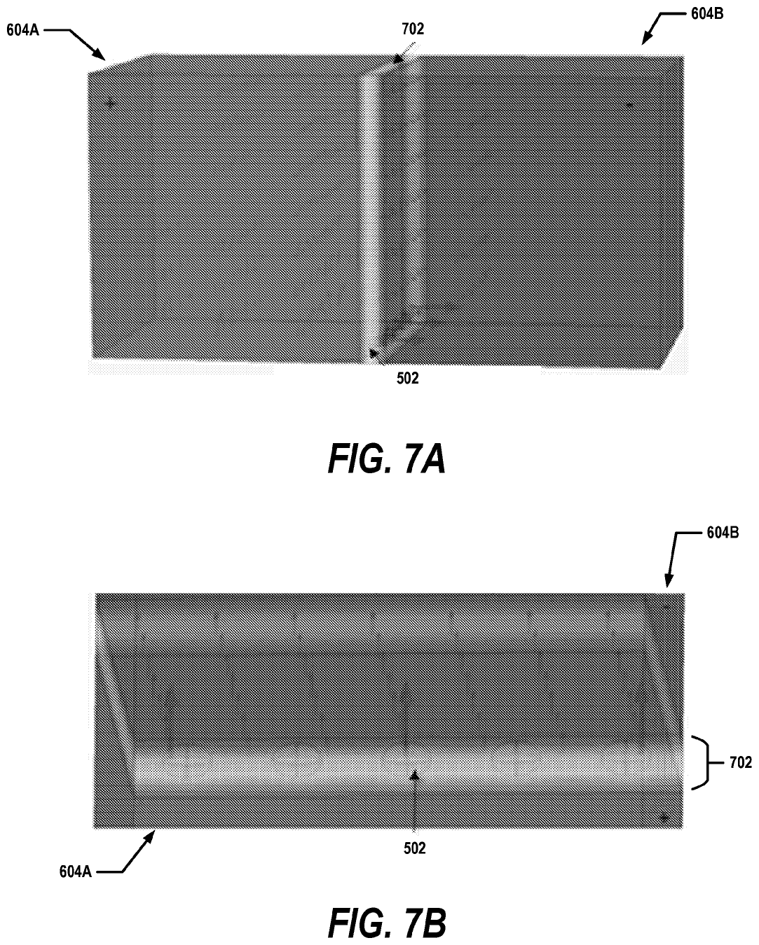

FIGS. 7A-B provide diagrams illustrating a uniformly distributed electric field being provided across a microfluidic channel 702. As shown and understood, electric current (e.g., indicated by arrows) flows from the positive electrode 604A to the negative electrode 604B, and the positive electrode 604A and the negative electrode 604B are positioned such that electric current flows through the microfluidic channel 702 to affect droplets 502, and EVs/exosomes within the droplets, positioned within the microfluidic channel. In the illustrated embodiment, the microfluidic channel 702 is approximately 1000 μm×1000 μm×100 μm, and both of the positive electrode 604A and the negative electrode 604B are configured to interface with at least a majority portion of the 1000 μm×1000 μm faces of the microfluidic channel. That is, in various embodiments, the positive electrode 604A and the negative electrode 604B are sized in order to cause electric current to flow through at least a majority of the microfluidic channel 702. As such, droplets continuously flowing through the microfluidic channel are subjected to the electric current at substantially the same time. It may be appreciated that configuration of the electrodes 604 on either side of the microfluidic channel and the positioning of EVs/exosomes 606 within droplets 502 significantly reduces any potential contamination compared to other techniques such as conventional cuvette-based electroporation (e.g., in which an electroporation instrument in directly inserted and contacts a cell culture).

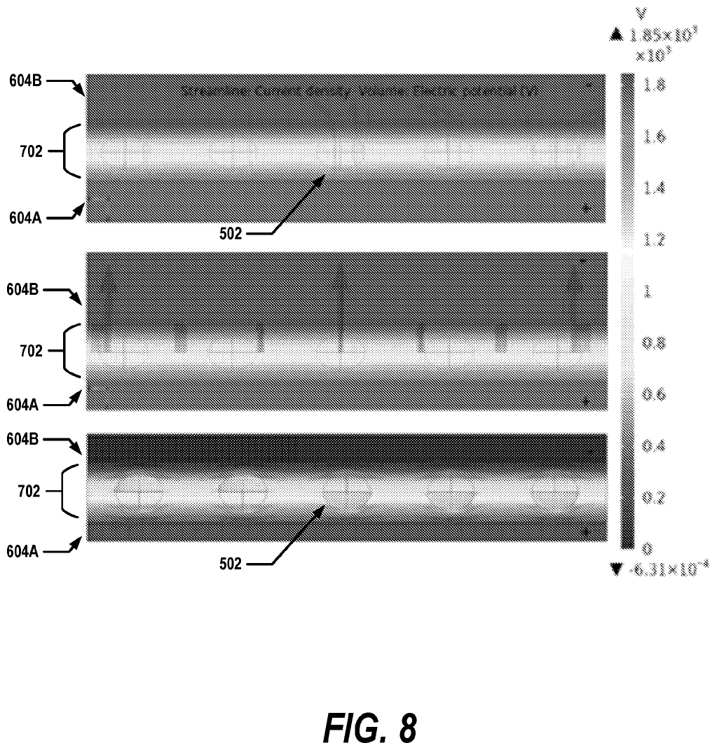

FIG. 8 provides a diagram illustrating electric potential distribution and electric current density across the microfluidic channel 702. The electric potential distribution and electric current density provided by the diagram may be simulated or modelled. For reference, example droplets 502 positioned within the microfluidic channel 702 are illustrated. As shown, electric potential is higher at the positive electrode 604A and decreases as a gradient approaching the negative electrode 604B. The diagram further illustrates streamlines and arrows indicating current density. As shown, electric current passes through the droplets 502 and affects the droplets 502.

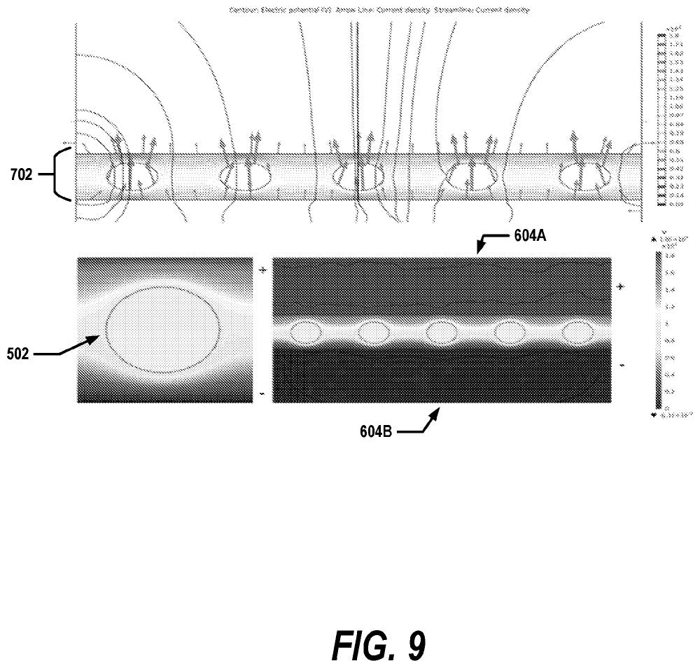

FIG. 9 provides another diagram also illustrating electric potential distribution and electric current density across the microfluidic channel 702. FIG. 9 further illustrates electric potential distribution near and within an example droplet 502. As shown in FIG. 9, electric current flows across the microfluidic channel 702. In particular, the diagram of FIG. 9 illustrates the conduction of a droplet 502. That is, while the electric potential may decrease in a gradient from the positive electrode 604A to the negative electrode 604B, the electric potential within droplet 502 may be relatively uniform. Due to conductivity of the droplet 502, EVs/exosomes 606 positioned through the droplet 502 may be affected by electric current and electroporated, such that biomolecular cargo 608 may enter the EVs/exosomes 606. An integrity of the EVs/exosomes 606 and membranes thereof is relatively maintained and undamaged from direct electric potential from the electrodes 604 and the joule heating issue, as diagram 900 illustrates that the electric potential within the droplet 502 is an average electric potential (e.g., an average between the electric potential associated with the positive electrode 604A and the negative electrode 604B).

Thus, with the electric field being configured to be uniformly distributed for a volume containing droplets 502 and providing an electric potential configured to cause electroporation of EVs/exosomes 606 positioned within droplets 502, the EVs/exosomes 606 may be electroporated and transfected with the biomolecular cargo 608 also positioned within the droplets 502. In various embodiments, a volume of droplets 502 continuously flows through the microfluidic channel, and the electric field is continuously provided across the microfluidic channel for the duration of droplet flow. In various embodiments, the electric field is a pulsed electric field, and pulses at a configurable frequency during the duration that droplets 502 continuously flow through the microfluidic channel. In various embodiments, the frequency at the electric field may pulse is based at least in part on the flow rate of the droplets 502 through the microfluidic channel, such that a particular droplet 502 flowing through the microfluidic channel is subjected to at least a number of electric field pulses. As such, a significant percentage of EVs/exosomes 606 are efficiently electroporated and loaded with the biomolecular cargo 608 (e.g., transfected).

Following electroporation of the EVs/exosomes, various subsequent actions may be performed. In various embodiments, loaded or transfected EVs/exosomes are extracted (purified or isolated) from the droplets. In some instances, transfected EVs/exosomes are separated from non-transfected or poorly transfected EVs/exosomes. In other instances, the entire population of EVs/exosomes 606 is gathered and harvested. In some instances, the population of EVs/exosomes 606 that are positioned in the droplets 502 are tested for successful loading (e.g., transfection), and an approximate fraction or ratio of successful loading is determined. Using the approximate fraction or ratio of successful loading (e.g., successful transfection from electroporation), various aspects of the EV/exosome transfection platform 130 may be configured and modified. As one example, a mass and/or volume ratio of EVs/exosomes 606 to biomolecular cargo 608 may be adjusted such that an increased amount of biomolecular cargo 608 is available to be transfected into the EVs/exosomes 606. In various embodiments, the post-electroporation EVs/exosomes are subsequently used in drug delivery applications and/or testing, as well as therapeutic applications and/or testing.

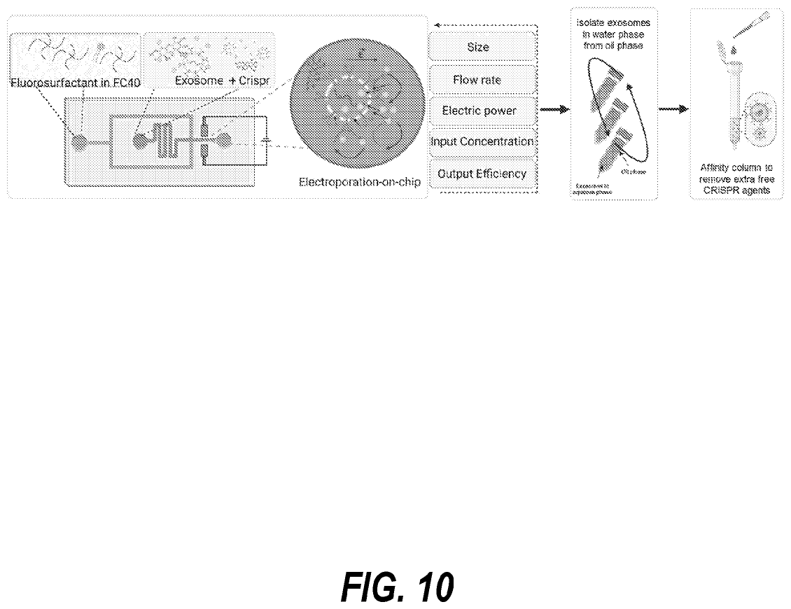

FIG. 10 provides another exemplary diagram illustrating a workflow for generating clustered regularly interspaced short palindromic repeats (CRISPR) EVs using optimized microfluidic droplet electroporation device, according to an example embodiment. As shown in FIG. 10, due to a higher mass transfer in a smaller volume and streamlined CRISPR electroporation in droplet, much lower voltage output may be used for scaling up. The cargo loaded EVs in the water phase can be collected easily downstream into a collection tube for membrane recovery. EV harvesting can be achieved by applying a centrifuge at 200 xg, 500 xg, and 2000 xg for ˜6 min respectively to fully separate the oil phase from the aqueous phase, which did not show any adverse impact on the resulted EVs. The extra free CRISPR reagents can be removed using an affinity column to obtain pure CRISPR loaded EVs.

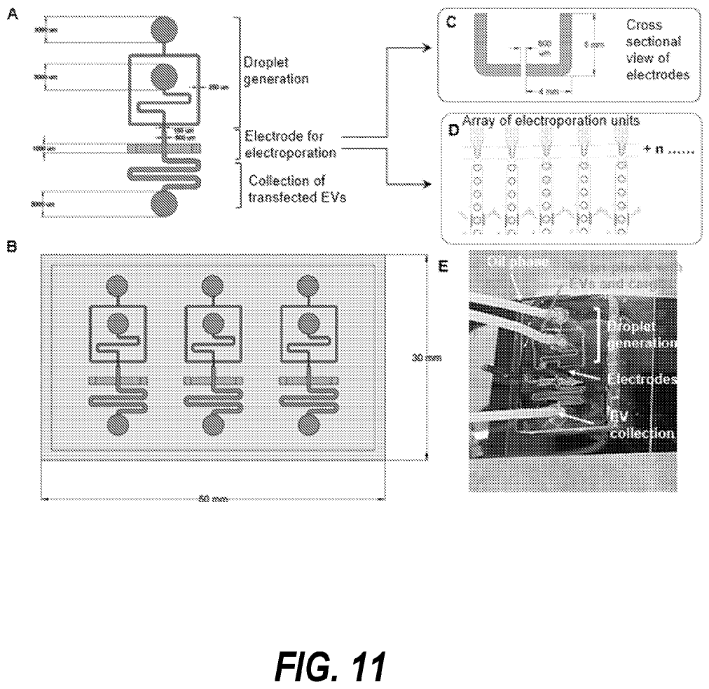

FIG. 11 is an exemplary diagram illustrating an engineering design of a microfluidic droplet electroporation device (μDES), according to an example embodiment. As shown in FIG. 11, section A demonstrates the engineering design of microfluidic droplet electroporation device (μDES). The microfluidic droplet electroporation device may be fabricated by using a 3-dimension printer to print a mold, and then using Polydimethylsiloxane (PDMS) replication method with the mold. Section B demonstrates using the PDMS polymer to replicate the structure to form the device. Section C demonstrates an exemplary diagram of the electrode dimension in a cross-sectional view. Section D demonstrates an exemplary diagram of an array format for multiplexed electroporation of multiple samples in parallel. Section E demonstrates a fabricated PDMS microfluidic droplet electroporation device (μDES) with functional illustration.

FIG. 12A is an exemplary diagram illustrating a picture of a PDMS fabricated device from a 3-dimension printed mold, according to an example embodiment.

FIG. 12B is an exemplary diagram illustrating a picture of a continuous-flow microfluidic droplet electroporation system (μDES) for harvesting cargo loaded EVs, according to an example embodiment.

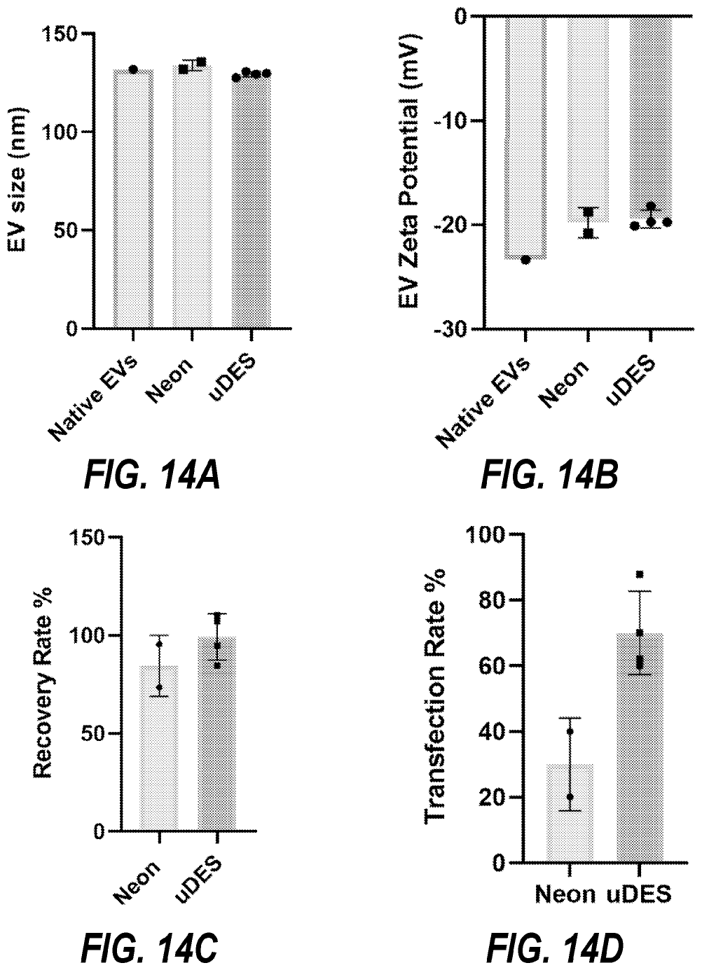

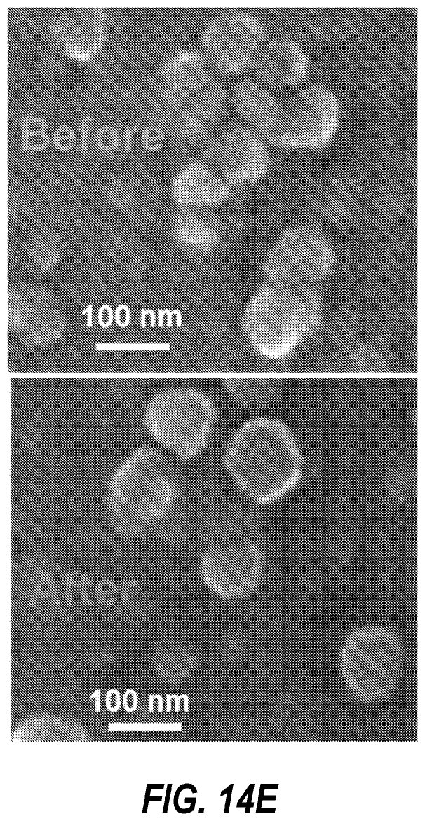

FIG. 14A provides an exemplary diagram illustrating a nanoparticle tracking analysis (NTA) of sizes and concentrations from HEI-OC1 EVs after μDES transfection, compared with conventional Neon system transfected EVs, and native EVs without electro-transfection, according to an example embodiment. As shown in FIG. 14A, the NTA indicates that the EVs have a consistent size of about 130 nm.

FIG. 14B provides an exemplary diagram illustrating a zeta potential analysis of μDES transfected EVs, compared with conventional Neon system transfected EVs, and native EVs without electro-transfection, according to an example embodiment.

FIG. 14C provides an exemplary diagram illustrating a recovery rate of μDES transfected EVs compared with conventional Neon system transfected EVs, according to an example embodiment. As shown in FIG. 14C, the recovery rate equals the number of the harvested CRISPR EV particles/the original number of input EV particles measured by NTA.

FIG. 14D provides an exemplary diagram illustrating a transfection rate of μDES transfected EVs compared with conventional Neon system transfected EVs, according to an example embodiment. As shown in FIG. 14D, the transfection rate equals the number of fluorescence CRISPR EV particles/the number of total CRISPR EV particles measured by fluorescence NTA and using EGFP-Cas9/gRNA RNP.

FIG. 14E provides an exemplary diagram illustrating transmission electron microscopy (TEM) images showing the morphology of HEI-OC1 EVs before electroporation and after μDES electroporation, according to an example embodiment.

Thus, example embodiments of the present disclosure provide for microfluidic droplet-based electroporation for EV/exosome cargo loading (e.g., EV/exosome transfection) and provide various technical advantages including improved efficiency, improved throughput of transfection, improved integrity of EVs/exosomes during electroporation, and reduced contamination. Due to the use of microfluidic channels and configuration of electrodes, multiple droplets that are positioned within a microfluidic channel can be simultaneously or almost simultaneously subjected to electroporation (e.g., subjected to electric currents configured to cause electroporation). With continuous flow of droplets through the microfluidic channel, throughput and efficiency of EV/exosome cargo loading (e.g., transfection) is significantly improved. Further, storage of EVs/exosomes and biomolecular cargo within droplets may control and/or limit electric currents affecting the EVs/exosomes such that integrity (e.g., of membranes) of the EVs/exosomes can be maintained following electroporation. Additionally, the storage of EVs/exosomes and biomolecular cargo within droplets reduces contamination that may occur with contact with the electrodes.

### V. Exemplary Use Case

As an exemplary use case of embodiments of the present invention, EV-delivered CRISPR/Cas9 into the inner ear blood-labyrinth barrier and inner ear hair cells can be used as the gene therapy in treating hearing loss. For example, embodiments of the present invention can use EVs derived from HEI-OCI hair cells to deliver CRISPR/Cas9 as the intact complexes to target the gene mutation at MYO7A. Such HEI-OCI hair cell-derived EVs are naturally presented between the blood-labyrinth barrier in the inner ear for cellular regulation. Thus, the CRISPR/Cas9 loaded HEI-OC1 EVs comprise a new platform for crossing inner ear blood labyrinth barrier in vivo, and for specifically targeting MYO7A mutation in the inner ear hair cells, which leads to higher safety and specificity for gene therapy.

EVs/exosomes produced using the described methods can be loaded with one or more cargo molecules. The cargo molecule can be, for example, a therapeutic molecule or targeting molecule. In some embodiments, the EVs/exosomes contain one or more nucleic acids such as, but not limited to, antisense oligonucleotides, siRNAs, miRNAs, and CRISPR RNAs. In some embodiments, the EVs/exosomes are loaded with one or more polypeptides. The polypeptides can be, but are not limited to, antigens, tumor antigens, pathogenic antigens, viral antigens, bacterial antigens, fungal antigens, parasitic antigens, cytokines, targeting ligands, and immune co-stimulators, an antibody, a cytokine, a growth factor, a hormone, or an enzyme. In some embodiments, the cargo loaded EVs/exosomes contain one or more drugs. Targeting molecules include, but are not limited to: MHC molecules, tetraspanin family proteins, integrin family proteins, cell receptors and cell receptor ligands. A cargo molecule is loaded onto an EV/exosome if it is present in the interior of the EV/exosomes, or in the membrane of the EV/exosome (e.g., integral membrane protein).

In some embodiments, the cargo loaded EVs/exosomes produced according to the described methods are used to deliver the cargo molecule to a cell or tissue. The cell or tissue can be in a subject. The subject can be a mammal, such as a human. In some embodiments, the cargo is a therapeutic molecule such as a drug.

In some embodiments, the cargo loaded EVs/exosomes produced according to the described methods are suitable for EV/exosome-based therapies and/or vaccines.

In some embodiments, the cargo loaded EVs/exosomes produced according to the described methods are suitable for EV/exosome-based regenerative therapies.

In some embodiments, the cargo loaded EVs/exosomes produced according to the described methods are used to modulate immune function in a subject, i.e., immunotherapy. The subject can be a mammal, such as a human.

In some embodiments, the cargo loaded EVs/exosomes produced according to the described methods are used to deliver a nucleic acid to a cell or tissue. The cell or tissue can be in a subject. The subject can be a mammal, such as a human.

In some embodiments, the EVs/exosomes produced according to the described methods are used in immunotherapy. In some embodiments, the EVs/exosomes produced by the methods described herein can be used to modulate an immune reaction or immunoregulation.

In some embodiments, methods of providing therapeutic extracellular vesicles to a subject are provided comprising: loading a therapeutic cargo molecule into an EV/exosome using any of the described devices or methods to form a cargo loaded EV/exosome, and administering the cargo loaded EV/exosome to the subject.

### VI. Exemplary Computer Program Product, Methods, and Computing Entities

Embodiments of the present disclosure may be implemented in various ways, including as computer program products that comprise articles of manufacture. A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described herein with reference to block diagrams and/or flowchart illustrations. Thus, it should be understood that each block of the block diagrams and/or flowchart illustrations may be implemented in the form of a computer program product, an entirely hardware embodiment, a combination of hardware and computer program products, and/or apparatus, systems, computing devices, computing entities, and/or the like, carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### VII. Conclusion

Many modifications and other embodiments of the present disclosure set forth herein will come to mind to one skilled in the art to which the present disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claim concepts. Although specific terms are employed herein, they are used in a generic and descriptive sense only and not for purposes of limitation.

1. A method for loading cargo into extracellular vesicles (EVs) and/or exosomes, the method comprising:
generating a plurality of droplets from a fluid comprising a biomolecular cargo and a plurality of EVs and/or exosomes; and
providing an electroporation potential to affect the plurality of droplets, a droplet configured to conduct an electrical current resulting from the electroporation potential to EVs and/or exosomes positioned within the droplet.

2. The method of claim 1, further comprising causing continuous flow of the plurality of droplets through a microfluidic channel across which the electroporation potential is provided.

3. The method of claim 2, wherein the electroporation potential is provided uniformly along the microfluidic channel such that droplets continuously flowing through the microfluidic channel are affected by the electroporation potential.

4. The method of claim 2, wherein the plurality of droplets is caused to continuously flow through the microfluidic channel for a time period, and wherein the electroporation potential is provided across the microfluidic channel for a duration of the time period.

5. The method of claim 1, wherein the electroporation potential is provided based at least in part on an electric field generated by at least one positive electrode and at least one negative electrode, the plurality of droplets being positioned between the at least one positive electrode and the at least one negative electrode.

6. The method of claim 1, further comprising extracting the plurality of EVs and/or exosomes from the plurality of droplets, at least some of the plurality of EVs and/or exosomes comprising the biomolecular cargo.

7. The method of claim 1, wherein the fluid is associated with a particular conductivity such that a droplet that is affected by the electroporation potential conducts an electric current to EVs and/or exosomes positioned within the droplet.

8. The method of claim 7, wherein the particular conductivity is between near 0 S/m and about 2 S/m.

9. The method of claim 1, wherein the electroporation potential is based at least in part on a threshold electroporation potential associated with the plurality of EVs and/or exosomes.

10. The method of claim 1, wherein the biomolecular cargo comprises one or more oligonucleotides, nucleic acids, plasmid, CRISPR agents, proteins, enzyme, antibodies, amino acids, carbohydrates, biomolecular or biochemical compounds, nanoparticles, magnetic particles, metal particles, cytokine, hormone, antigen, growth factor, drugs, therapeutic molecules, and/or active pharmaceutical ingredients.

11. The method of claim 1, wherein each of the plurality of droplets has a diameter of approximately 10 micrometers to approximately 3000 micrometers.

12. The method of claim 1, wherein the EVs and/or exosomes are obtained from a cell culture, blood, plasma, a tissue fluid, urine, milk, bacterial fluids, plant fluids, ascites, milk, a plant, a plant cell, or a bacterium.

13. An extracellular vesicle and exosome (EV/exosome) transfection platform comprising:
a droplet generation subsystem configured to generate a plurality of droplets from a fluid comprising a biomolecular cargo and a plurality of EVs and/or exosomes;
an electrical field subsystem comprising a positive electrode and a negative electrode configured to generate an electric field across the plurality of droplets; and
a controller configured to control operation of the droplet generation subsystem and the electrical field subsystem, wherein the controller is configured to operate the EV/exosome
transfection platform to produce a population of EVs and/or exosomes comprising the biomolecular cargo.

14. The EV/exosome transfection platform of claim 13, further comprising a microfluidic flow subsystem comprising at least one microfluidic channel configured for continuous flow of the plurality of droplets that are generated, the controller configured to control operation of the microfluidic flow subsystem.

15. A computer program product for controlling an extracellular vesicle and exosome (EV/exosome) transfection platform, the computer program product comprising at least one non-transitory computer-readable storage medium storing computer executable code portions, the computer executable code portions comprising computer executable instructions configured to, when executed by a processing element of an apparatus, cause the apparatus to cause:
a droplet generation subsystem of the EV/exosome transfection platform to generate a plurality of droplets from a fluid comprising a biomolecular cargo and a plurality of EVs and/or exosomes;
an electrical field subsystem of the EV/exosome transfection platform to generate an electric field across the plurality of droplets, wherein the electrical field subsystem comprises a positive electrode and a negative electrode; and
a controller configured of the EV/exosome transfection platform to control operation of the droplet generation subsystem and the electrical field subsystem, wherein the controller is configured to operate the EV/exosome transfection platform to produce a population of EVs and/or exosomes comprising the biomolecular cargo.

16. The computer program product of claim 15, wherein the apparatus is a controller of an EV/exosome transfection platform or a computing entity in communication with the controller of the EV/exosome transfection platform.

17. An EV and/or exosome made by the method of claim 1.

18. The EV and/or exosome of claim 17, for use in delivering or providing a therapeutic treatment to a subject.

19. The EV and/or exosome of claim 18, wherein providing a therapeutic treatment to a subject comprises: immunotherapy, gene therapy, or regenerative therapy.

20. A method of providing therapeutic EV and/or exosome to a subject comprising: loading a therapeutic cargo molecule into an EV/exosome using the method of claim 1 to form a cargo loaded EV and/or exosome, and administering the cargo loaded EV and/or exosome to the subject.

21. A method of immunotherapy, gene therapy, or regenerative therapy comprising: loading a therapeutic cargo molecule into an EV and/or using the method of claim 1 to form a cargo loaded EV and/or exosome, and administering the cargo loaded EV and/or exosome to the subject.
